(19) United States
(12) Patent Application Publication
(10) Pub. No.: US 2009/0305403 A1
(43) Pub. Date: Dec. 10, 2009

(54) Isolation and Differentiation of Adult Hippocampal Arctic Squirrel Neural Stem Cells

(75) Inventor: Judith Kelleher-Andersson, Clarksville, MD (US)

Correspondence Address: Davis, Agnor, Rapaport & Skalny, LLC, 10211 Wincopin Circle, Suite 600, Columbia, MD, 21044, US

(73) Assignee: Neuronascent, Inc., Clarksville, MD (US)

(21) Appl. No.: 12/439,218

(22) PCT Filed: Aug. 31, 2007
(86) PCT No.: PCT/US07/77355

§ 371 Date: Feb. 27, 2009

Related U.S. Application Data
(60) Provisional application No. 60/824,321, filed on Sep. 1, 2006.

(51) Int. Cl. C12N 5/10; C12N 5/06; C07K 16/18

(52) U.S. Cl. 435/352; 435/377; 530/387.9

(57) ABSTRACT

Neuronal stem cell lines derived from the Arctic Ground Squirrel, methods related to culturing and maintaining a neuronal stem cell line derived from the Arctic Ground Squirrel and a culture media required to maintain and differentiate a neuronal stem cell line derived from the Arctic Ground Squirrel is disclosed. Antibodies specific for antigens expressed on a neuronal stem cell line derived from the Arctic Ground Squirrel, and products and methods related to the use of neuronal stem cell lines derived from the Arctic Ground Squirrel are also included.

### GOVERNMENT INTERESTS

Certain research which gave rise to the present invention was supported by SNRP-NIH Grant NS41069. Consequently, the government may retain certain rights in the invention.

### BACKGROUND

1. Field of the Invention

This invention relates to the culture, maintenance and differentiation of neuronal and/or neural stem cells obtained from adult Alaskan Ground Squirrel or better known as Arctic Ground Squirrels (Spermophilus parryii).

2. Background of the Invention

The Arctic ground squirrel (Spermophilus parryii) is found in Alaska, Canada in the Yukon Territory, northern British Columbia, and the mainland of the Northwest Territory, among others. The Arctic ground squirrel lives in alpine and arctic tundra in meadows, riverbanks, lakeshores, and sandbanks. The Arctic ground squirrel is an herbivore in the summer, it begins to store willow leaves, seeds and grasses in its burrow. The Arctic ground squirrel hibernates for up to seven months, from September through April and uses the store of food after it wakes up in the spring.

Over the course of the hibernation season, the animal experiences extreme decreases in body temperature, heart rate, blood flow and oxygen consumption, which is interrupted at periodic intervals when animals spontaneously re-warms to 37° C. about every 5 to 14 days. These short periods of warm body temperature last 24-48 hrs. During these repeated warming periods, Arctic ground squirrels are able to meet the increased energy demands even though blood flow, oxygen and glucose delivery plummet by as much as 90%. Several physiological changes associated with hibernation include hypothermia, increased clotting times, increased ascorbate blood concentrations and decreased leukocyte counts. One hypothesis is that elevated levels of ascorbate in plasma acts as an antioxidant during the metabolic stress that accompanies arousal from hibernation (Tøien Ø. et al., Am. J. Regulatory Integrative Comp. Physiol. 281: R572-R583, 2001).

It is known in the art that the Arctic ground squirrel is useful as a model for stroke. (see: www.gi.alaska.edu/ScienceFuorum/ASF13/1378, 1998; Boyer B F B and Barnes B M (1999), Bioscience 49: 713-724; Lyman C P, et al. Hibernation and torpor in mammals and birds. Academic Press, New York, 1982; Becker L B, et al., Circulation 105:2562-2570, 2002), ischemia-reperfusion, traumatic brain injury and neurodegenerative diseases (Drew, K L, et al., Free Rad. Biol. Med., 31(5):563-573, 2001) and for neuroprotection (Zhou F, et al., Am. J. Pathol. 158:2145-2151, 2001).

Stroke can be caused by an interruption of cerebral blood flow. Only tissue plasminogen activator (TPA) has proven effective in controlled clinical trials. While TPA has improved prognosis for many patients, progress in the development of effective therapies has been slow.

The pathological events in Alzheimer's disease and late onset dementia may be triggered by or, at minimum, exacerbated by, impaired cerebral perfusion originating in the microvasculature which affects the optimal delivery of glucose and oxygen and results in a breakdown of metabolic energy pathways in brain cells. Oxidative stress, the imbalance between processes leading to free radical production and the cellular antioxidant cascade, is likewise, intimately associated with the neurodegenerative process. Hypoperfusion with subsequent disruption of energy balance and ion homeostasis and initiation of a cascade of events that ultimately leads to oxidative stress and cell death may thus be a common factor in neurodegeneration following stroke and in neurodegenerative disease. Disruption of energy balance and ion homeostasis following traumatic brain injury (TBI), likewise, initiates a cascade of inflammation, toxicity and oxidative stress that exacerbates acute brain tissue trauma.

U.S. Pat. No. 6,251,669 B1 describes a culture method for rat neuronal progenitor cells.

There exists a need in the art for a reliable animal model to facilitate the development of therapies for stroke, hypoxia, and other brain injuries, as well as the need for a model of neurogenesis.

### SUMMARY OF THE INVENTION

The present invention is drawn to neuronal stem cell lines derived from the Arctic Ground Squirrel.

The invention is also directed to methods related to culturing, maintaining and differentiating neuronal stem cell lines derived from the Arctic Ground Squirrel.

The invention also includes culture media required to maintain and differentiate a neuronal stem cell line derived from the Arctic Ground Squirrel.

The invention is further directed to specific antibodies specific for antigens expressed on a neuronal stem cell line derived from the Arctic Ground Squirrel.

Methods related to the use of a neuronal stem cell line derived from the Arctic Ground Squirrel are also included. Said methods include, but are not limited to: transplantation into brain of stroke-susceptible rodentia species such as a rat; discovery of genes and/or proteins key to hibernation; discovery of genes and/or proteins key to neuroprotection; discovery of genes and/or proteins key to neurogenesis; and/or testing of potential therapeutics that may mimic hibernation, ischemic tolerance and neurogenesis; determination and looking at fate changes of the stem cells to neuron lineages.

Methods related to the use of a neuronal progenitor cells derived from the Arctic Ground Squirrel are also included. Said methods include, but are not limited to: transplantation into brain of stroke-susceptible species such as a rat; discovery of genes and/or proteins key to hibernation; discovery of genes and/or proteins key to neuroprotection; discovery of genes and/or proteins key to neurogenesis; and/or testing of potential therapeutics that may mimic hibernation, ischemic tolerance, and neurogenesis; determination and looking at fate changes of the stem cells to neuron lineages.

Products related to the use of a neuronal stem cell line derived from the Arctic Ground Squirrel are also included in the present invention. Said methods include, but are not limited to: transplantation into brain of stroke-susceptible species (rat); discovery of genes and/or proteins key to hibernation; discovery of genes and/or proteins key to neuroprotection; discovery of genes and/or proteins key to neurogenesis; and/or testing of potential therapeutics that may mimic hibernation, ischemic tolerance and neurogenesis; determination and looking at fate changes of the stem cells to neuron lineages.

### BRIEF DESCRIPTION OF THE FIGURES

FIG. 1 depicts Nestin-positive AGS hippocampal neural stem cells.

FIG. 2 shows neuron-specific Class III β-tubulin-positive cells.

FIG. 3 depicts AGS neurons under normoxia and hypoxia conditions.

### DETAILED DESCRIPTION OF THE INVENTION

This invention includes the culture, maintenance and differentiation of neuronal and neural stem cells obtained from adult Alaskan Ground Squirrel or better known as Arctic Ground Squirrels with these terms being equivalent and are interchangeable. The example of the Arctic ground squirrel covered here is Spermophilus parryii.

As used herein, “neuronal cells” or “neurons” includes cells which are post-mitotic and which express one or more neuron-specific markers. Examples of such markers can include but are not limited to neurofilament, microtubule-associated protein-2, and tau, and preferably neuron-specific Class III β-tubulin and specific neuron marker, NeuN used herein “neuronal progenitor cells” are cells which can give rise to progeny which can differentiate into neuronal cells, but, unlike neuronal cells, are capable of cell division in vivo or in vitro, and which also, like post-mitotic neurons, express a neuron-specific marker.

In these compositions, preferably only about 40%, or more preferably about 10%, or even more preferably about 5%, or fewer of the cells in the composition are non-neuronal cells. Non-neuronal cells include cells which express a glia-specific marker, such as glial fibrillary acidic protein (GFAP), or which do not express any neuron-specific markers. Non-neuronal cells can include but are not limited to glial cells, subependymal cells, and fibroblasts and do not include neuronal progenitor cells.

As used herein, the “progeny” of a cell can include any subsequent generation of the cell. Thus, the progeny of a neuronal progenitor cell can include, for example, a later generation neuronal progenitor cell, a later generation cell that has undergone differentiation, or a fully differentiated, post-mitotic neuronal cell.

The present invention provides a cellular composition comprising mammalian, non-tumor derived cells which express a neuron-specific marker and which can divide. The cellular composition can be isolated from the region corresponding to the hippocampus region of AGS brain as described further herein and exemplified in the Examples below. The substantially homogeneous composition can be obtained in the absence of treatment with mitotic inhibitors. In addition, the ability of the cells to divide can be achieved in the absence of immortalization techniques. The neuronal stem cells can, without being first immortalized, divide for at least two generations. At least about two, preferably at least about five, and more preferably at least about eight or more generations of differentiated neurons can result when the isolated cells are placed in standard culture conditions as exemplified in the Examples below.

Additionally, the cells of the substantially homogeneous composition of neuronal progenitor cells can give rise to progeny which can differentiate into neuronal cells. By use of this composition, therefore, one can obtain, in the absence of mitotic inhibitors, a composition comprising greater than 60%, and preferably greater than 90%, and more preferably greater than 95%, of any of the following cells: neuronal progenitor cells, progeny of neuronal progenitor cells and neuronal cells.

Additionally, the cells present invention provides a cellular composition comprising mammalian non-tumor derived neural stem cells, which can divide and differentiate to the cells of the nervous system.

The cells comprising the herein described composition can be isolated from the hippocampus or cortex of the brain of the AGS.

“Neuronal progenitor,” as used herein is defined as a cellular composition of greater than about 90% mammalian, non tumor-derived, neuronal progenitor cells which express a neuron-specific marker and which can give rise to progeny which can differentiate into neuronal cells.

“Neural Stem cell” as used herein is defined as a cellular composition that are self-renewing and multi-potential. Neural stem cells are known to express nestin, an intermediate filament. These Neural stem cells are capable of differentiating to neurons and macroglia (e.g. astrocytes and oligodendrocytes). The present invention provides a cellular composition wherein at least a portion of the cells can be transfected by a selected nucleic acid. The cells can be transfected with an exogenous nucleic acid as exemplified in the Examples below. “Exogenous” can include any nucleic acid not originally found in the cell, including a modified nucleic acid originally endogenous to the cell prior to modification. By “transfected” is meant to include any means by which the nucleic acid can be transferred, such as by infection, transformation, transfection, electroporation, microinjection, calcium chloride precipitation or liposome-mediated transfer. These transfer methods are, in general, standard in the art (see, e.g., Sambrook et al., Molecular Cloning: A Laboratory Manual, 2nd Ed., Cold Spring Harbor Laboratory, Cold Spring Harbor, N.Y. (1989)). Preferably at least about 3%, more preferably about 10%, more preferably about 20%, more preferably about 30%, more preferably about 50%, and even more preferably about 75% of the cells, at least initially after transfection, are transfected. To increase the percentage of transfected cells, multiple transfections can be performed. For example, one can infect cells with a vector of choice, remove the media after infection, reinfect, etc. and repeat the process to achieve the desired percentage of infected cells. Some viruses, for example, can be viable for about two hours at a 37° C. incubation temperature; therefore, the infection can preferably be repeated every couple of hours to achieve higher percentages of transfected cells. Other methods of increasing transfected cell number are known and standard in the art.

Any selected nucleic acid can be transferred into the cells. For example, a nucleic acid that functionally encodes a biologically active molecule can be transfected into the cells. Preferably nucleic acids can include, for example, nucleic acids that encode a biologically active molecule that stimulates cell division or differentiation such as, for example, growth factors, e.g., nerve growth factor (NGF), brain-derived neurotrophic factor (BDNF), neurotrophin (NT)-3 and NT4/5, ciliary neurotrophic factor (CNTF), and factors that block growth inhibitors. Additionally, preferable nucleic acids can include nucleic acids that encode a biologically active molecule that functions in the synthesis of a neurotransmitter. The nucleic acid can be in any vector of choice, such as a plasmid or a viral vector, and the method of transfer into the cell can be chosen accordingly. As known in the art, nucleic acids can be modified for particular expression, such as by using a particular cell- or tissue-specific promoter, by using a promoter that can be readily induced, or by selecting a particularly strong promoter, if desired.

Cell lines of the invention include cell lines obtained wherein the gene target or targets for ischemic tolerance in Arctic Ground Squirrels has been knocked out. The methods to obtain “knock out” genes include using RNAi (RNA interference) technologies. RNA interference may encompass the introduction of homologous double stranded RNA (dsRNA) to specifically target a gene's product, resulting in null or hypomorphic phenotypes. The use of antisense RNA to interfere with a gene's activity was first utilized in C. elegans and later it was reported that control sense RNA also could produce a mutant phenotype (Cell 81: 611-20, 1995). Subsequently, it was discovered that it is the presence of dsRNA, formed from the annealing of sense and antisense strands present in the in vitro RNA preparation, that is responsible for producing the interfering activity. Introduction of dsRNA into an adult worm results in the loss of the targeted endogenous mRNA from both the adult and its progeny. Long double-stranded RNAs (dsRNAs; typically >200 nt) can be used to silence the expression of target genes in a variety of organisms and cell types (e.g., worms, fruit flies, and plants).

Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand.

In mammalian cells, it has been shown, that for example, that introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs, therefore, the system has been shown to be present in mammalian systems as well (Applied Biosytems, Inc., Ambion Online Appendix, 2007).

The invention includes knock out gene in Arctic Ground Squirrel cells and/or cell lines, and also includes Arctic Ground Squirrel cells and/or cell lines which contain said knock out genes. Said knock our genes include gene targets for ischemic tolerance in Arctic Ground Squirrels.

The present invention also provides methods for isolating the cellular compositions. Thus, methods are provided for isolating a substantially homogeneous composition using special culture conditions in the absence of mitotic inhibitors. Specifically, the present invention provides a method of obtaining an isolated cellular composition wherein greater than about 90%, and preferably greater than about 95%, and even more preferably greater than about 98%, of the cells of the composition are non-tumor-derived, neuronal progenitor cells which express a neuronal marker and which can give rise to progeny which can differentiate into neuronal cells, comprising isolating cells from the hippocampus or cortex of an AGS brain and culturing the cells in the absence of mitotic inhibitors. Thus, the cellular composition, as isolated, can be substantially devoid (i.e., comprises less than 10%, preferably less than 5%, more preferably less than 2%) of glial and other non-neuronal cells, and thus culture conditions designed to eliminate non-neuronal cells from the compositions can often be omitted. Therefore, the cultured cells are not subjected, for example, to mitotic inhibitors. However, if desired, mitotic inhibitors can be utilized.

The present invention also provides a method of screening for markers of neuronal cells. Specifically, the present invention provides a method of screening for a marker of neuronal cells comprising obtaining the cellular composition described herein (which composition comprises greater than about 90% or 95% neuronal progenitor cells which express a neuron-specific marker and which can give rise to progeny which can differentiate into neuronal cells), obtaining non-neuronal cells or information concerning the markers of those cells, and detecting the presence of a marker in the cellular composition that is not present in non-neuronal cells, the marker present in the cellular composition that is not present in the non-neuronal cells being a marker of neuronal cells, especially if those markers are found specifically in Arctic ground squirrel. Thus, markers of the cellular composition can be compared to markers of non-neuronal cells to identify markers present in neurons, exclusively or in greater proportions. Markers of this cellular composition can be compared to non-hibernating animal neuron cultures to identify markers present in neurons exclusively or in greater or lesser proportions in the AGS squirrels. These AGS-specific markers can be useful in diagnostic and therapeutic techniques for neuronal diseases. The neuron-specific markers can be useful in diagnostic and therapeutic techniques for neuronal diseases.

Additionally, the present invention provides a method of detecting a neuronally expressed gene comprising obtaining a cDNA library from the herein described cellular composition for determining genes specific to the hibernation capacity of Arctic ground squirrels, obtaining a cDNA library from a non-neuronal cell, determining the presence at higher or lower levels of a cDNA in the library from the cellular composition than in the non-neuronal cell of other non-hibernating species, the presence at higher levels of a cDNA in the library from the cellular composition indicating a neuronally expressed gene. Methods of performing such comparative screenings are known in the art, and thus can be readily performed by the artisan given the teachings herein. The AGS neuron-specific genes, gene products, and/or markers could be useful in diagnostic and therapeutic techniques for neuronal diseases.

By the tern “gene” is meant a segment of DNA which encodes a specific protein or polypeptide, or RNA. The term “gene” includes not only coding sequences but also regulatory regions such as promoter, enhancer and terminator regions. The term further includes all introns and other DNA sequences spliced from the final gene RNA transcript. Further, the term includes the coding sequences as well as the non-functional sequences. All DNA sequences provided herein are understood to include complementary strands unless otherwise noted. It is understood that an oligonucleotide may be selected from either strand of the genomic or cDNA sequences. Furthermore, RNA equivalents can be prepared by substituting uracil for thymine, and are included in the scope of this definition, along with RNA copies of the DNA sequences of the invention isolated from cells. The oligonucleotide of the invention can be modified by the addition of peptides, labels, and other chemical moieties and are understood to be included in the scope of this definition.

The term “polynucleotide” means any single-stranded sequence of nucleotide units connected by phosphodiester linkages, or any double-stranded sequences comprising two such complementary single-stranded sequences held together by hydrogen bonds. Unless otherwise indicated, each polynucleotide sequence set forth herein is presented as a sequence of deoxyribonucleotides (abbreviated A, G, C and T). However, the term “polynucleotide” is intended to mean a DNA molecule or polynucleotide, a sequence of deoxyribonucleotides, or an RNA molecule or polyribonucleotide. The corresponding sequence of ribonucleotides includes the bases A, G, C and U, where each thymidine deoxyribonucleotide (T) in the specified deoxyribonucleotide sequence is replaced by the ribonucleotide uridine (U).

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present invention includes an antibody which selectively binds a polypeptide of protein expressed by the Arctic ground squirrel cell lines described in the present invention. As used herein, an antibody “selectively binds” a target peptide when it binds the target peptide and does not significantly bind to unrelated proteins. The term “selectively binds” also comprises determining whether the antibody selectively binds to the target mutant sequence relative to a native target sequence. An antibody which “selectively binds” a target peptide is equivalent to an antibody which is “specific” to a target peptide, as used herein. An antibody is still considered to selectively bind a peptide even if it also binds to other proteins that are not substantially homologous with the target peptide so long as such proteins share homology with a fragment or domain of the peptide target of the antibody. In this case, it would be understood that antibody binding to the peptide is still selective despite some degree of cross-reactivity. In another embodiment, the determination whether the antibody selectively binds to the mutant target sequence comprises: (a) determining the binding affinity of the antibody for the mutant target sequence and for the native target sequences; and (b) comparing the binding affinities so determined, the presence of a higher binding affinity for the mutant target sequence than for the native indicating that the antibody selectively binds to the mutant target sequence.

The invention is further drawn to an antibody immobilized on an insoluble carrier comprising any of the antibodies disclosed herein. The antibody immobilized on an insoluble carrier includes multiple well plates, culture plates, culture tubes, test tubes, beads, spheres, filters, electrophoresis material, microscope slides, membranes, or affinity chromatography medium.

The invention also includes labeled antibodies, comprising a detectable signal. The labeled antibodies of this invention are labeled with a detectable molecule, which includes an enzyme, a fluorescent substance, a chemiluminescent substance, horseradish peroxidase, alkaline phosphatase, biotin, avidin, an electron dense substance, and a radioisotope, or any combination thereof.

The invention further includes a method of producing a hybridoma comprising fusing a mammalian myeloma cell with a mammalian B cell that produces a monoclonal antibody which selectively binds an amino acid sequence expressed on the surface of the cell lines disclosed herein, and a hybridoma producing any of the monoclonal antibodies disclosed herein. The invention further includes a method of producing an antibody comprising growing a hybridoma producing the monoclonal antibodies disclosed herein in an appropriate medium and isolating the antibodies from the medium, as is well known in the art. The invention also includes the production of polyclonal antibodies comprising the injection, either one injection or multiple injections of any of the polypeptides of the inventions into any animal known in the art to be useful for the production of polyclonal antibodies, including, but not limited to mouse, rat, hamster, rabbit, goat, sheep, deer, guinea pig, or primate, and recovering the antibodies in sera produced therein. The invention includes high avidity or high affinity antibodies produced therein. The invention also includes B cells produced from the listed species to be further used in cell fusion procedures for the manufacture of monoclonal antibody-producing hybridomas as disclosed herein.

The invention is further drawn to a kit comprising the antibody or a portion thereof as disclosed herein, a container comprising said antibody and instructions for use, a kit comprising the polypeptides of this invention and instructions for use and a kit comprising the polynucleotide of the invention, a container comprising said polynucleotide and instructions for use, or any combinations thereof. These kits would include, but not be limited to nucleic acid detection kits, which may, or may not, utilize PCR and immunoassay kits. Such kits are useful for clinical diagnostic use and provide standardized reagents as required in current clinical practice. These kits could either provide information as to the presence or absence of mutations prior to treatment or monitor the progress of the patient during therapy. The kits of the invention may also be used to provide standardized reagents for use in research laboratory studies.

Also provided by the present invention is a method of screening for a marker of Arctic ground squirrel neuronal cells comprising obtaining the neuronal progenitor cells of a cellular composition comprising greater than about 90% mammalian, non tumor-derived, neuronal progenitor cells which express a neuron-specific marker and which can give rise to progeny which can differentiate into neuronal cells, and detecting the presence of a marker in the neuronal progenitor cells that is not present in non-neuronal cells, the marker present in the neuronal progenitor cells that is not present in the non-neuronal cells being a marker of Arctic ground squirrel neuronal cells.

The present invention also provides a method of detecting a neuronally expressed gene comprising obtaining a cDNA library from the Alaskan ground squirrel neuronal progenitor cells of a cellular composition comprising greater than about 90% mammalian, non tumor-derived, neuronal progenitor cells which express a neuron-specific marker and which can give rise to progeny which can differentiate into neuronal cells, obtaining a cDNA library from a non-neuronal cell, determining the presence at higher levels of a cDNA in the library from the neuronal progenitor cells than in the non-neuronal cell, the presence at higher levels of a cDNA in the library from the neuronal progenitor cells indicating a neuronally expressed gene.

The present invention is drawn to the isolation, culture and maintenance of Arctic ground squirrel (AGS; also known as the Alaskan ground squirrel, Spermophilus parryii) adult stem cells. Arctic ground squirrel (AGS) adult stem cells are useful for scientists working with hibernation. The federal government; NIH, DARPA, and the Military has funded and been interested in hibernation previously. Government supported research to both induce suspended animation and to minimize battlefield injury have looked at hibernation as a model system.

The adult AGS stem cells are also useful for, but not limited to researchers and clinicians working in the area of stroke, hypoxia, other brain injuries and neurogenesis. The AGS stem cells are also useful for: transplantation into brain of stroke-susceptible species such as a rat; discovery of genes and/or proteins key to hibernation; discovery of genes and/or proteins key to neuroprotection; discovery of genes and/or proteins key to neurogenesis; and/or testing of potential therapeutics that may mimic hibernation/neurogenesis; determination and looking at fate changes of the stem cells to neuron lineages.

The Adult Hippocampal and Cortical AGS Differentiated Stem Cells are useful for the basic researcher interested in stroke, hypoxia, other brain injuries and neurogenesis related especially to hippocampus or cortex.

Using the differentiated neurons researchers can: measure differences in electrophysiologic output, including LTP; compare signal pathways between adult rat and AGS differentiated neural stem cells; compare susceptibility to hypoxia/brain injury between adult rat and AGS differentiated neural stem cells; and or for discovery of protein and gene targets for development of therapeutics for stroke, brain injury, or cognitive impairment.

Upon staining, the percentage of the neural stem cells that will become neurons or other (glia) cells can be determined. Differentiated neurons are also tested under hypoxic and low glucose conditions that partially mimic stroke. Rat adult hippocampal neural stem cells (Chemicon, Temecula, Calif.) are compared with AGS cells to show the usefulness and the “inherent” protective nature of the AGS cells. Various media formulations that are most appropriate for the AGS stem cells especially during the differentiation process are used. Media formulations that allow lineage differences of neurons are also included herein.

In order to prepare a culture medium for neural stem cells, hippocampus or cortex are collected from the anesthetized animal brain. The tissue is dissociated using techniques that may include but are not limited to trypsinization, proteolysis and centrifugation then are grown in media that optimizes proliferation of the cells. The neural stem cells are preferably collected from the brains of adult animals that have been in captivity and may or may not have been induced to hibernate in captivity.

Briefly, the cerebrum is excised out of the brain of an anesthesized adult animal and after the cerebral meninges is removed therefrom, the hippocampus cells are dissociated through use of enzymes such as trypsin, disperse, collagenase, and papain.

The collected neural stem cells can be cultured and proliferated in a medium containing animal serum. The animal serum is preferably bovine serum, and more preferably, fetal calf serum, calf serum, or neonatal calf serum. The amount of animal serum to be added is preferably in the range of 5-20%. In another embodiment the neural stem cells can be cultured in a serum free or chemically-defined condition. The medium is not particularly limited so long as it is a trophic medium for culturing animal cells. Examples of such medium include Neurobasal media, Eagle's minimum essential medium (hereinafter abbreviated as MEM), Dulbecco's modified Eagle medium (hereinafter abbreviated as DMEM), DMEM/HAM's F-12 medium (hereinafter abbreviated as F-12), F-12, and HAM's F-10 medium (hereinafter abbreviated as F-10), or any combination thereof. For example, although the mixing ratio of DMEM to F-12 in DMEM/F-12 media may vary, the ratio can be preferably in the range from 60/40 to 40/60 (on a weight basis) so that the resultant media have the traits of both media. Antibiotics and antimicrobials may be added to the medium to prevent microbial growth.

These media are often supplemented with insulin and transferrin. Preferably, selenious acid or a salt thereof is additionally incorporated. Insulin is added in an amount so as to achieve an insulin concentration of 1-100 μg/ml, and preferably 3-20 μg/ml. Transferrin is added in an amount so as to achieve a transferrin concentration of 1-100 μg/ml, and preferably 3-20 μg/ml. Examples of salts of selenious acid include sodium selenite and potassium selenite. It is preferred that selenious acid or a salt thereof be added in such an amount that will make its concentration 1-100 nM; particularly preferably 3-50 μM.

The neuronal cells dispersed in a culture medium are cultured on a flask, a dish, or a plate—all of which are used for cell culture—or a polylysine-coated flask, a polylysine-coated dish, a polylysine-coated plate, or a polylysine-coated microcarrier. In subculturing, a culture area of 4-50 times is preferred. Neural stem cells and neurons are confirmed by immunocytochemical staining. The neural stem cells stain positive for Nestin and neurons stain for any number of neuronal markers including microtubule-associated protein-2, and tau, neuron-specific Class III β-tubulin and specific neuron marker, NeuN.

To culture neurons from the neural stem cells, animal serum or mitogenic factors are removed. Neurons cannot be easily maintained and survive and other agents are then often necessary. Preferably, the culture supernatant additionally contains a combination of superoxide dismutase and catalase and/or α-tocopherols. Preferred concentrations of these supplements are 1-100 μg/ml for superoxide dismutase, 1-100 μg/ml for catalase, and 1-100 μg/ml for α-tocopherols. Examples of a-tocopherols include α-tocopherol and esters of α-tocopherol such as tocopherol acetate and tocopherol succinate. Other additives to the culture supplement may be retinoic acid, glutamate, a-tocopherol, progesterone and forskolin, or any other supplements known in the art. A preferable media may be Neurobasal (Invitrogen) media or other neuron specific medium. Also, the culture media of the invention must successfully overcome the problem of unstable culture which cannot be avoided when low-density culture is performed with some culture media.

The present invention includes a media which is specialized for the growth and maintenance to AGS cells. In order to incorporate the above-described supplements into a culture supernatant, a method similar to that described above may be used.

The present invention includes the culture supernate prepared from neuronal progenitors or neurons or astrocytes isolated originally from AGS as described. A culture supernatant can be stored stably in a frozen state. Therefore, in the case in which supernatants are collected every day consecutively, the supernatant collected each time is frozen without being combined with supplements such as insulin; and after a plurality of supernatants have been collected and frozen, they are thawed, uniformly mixed, and then combined with the supplements. This procedure provides more uniform culture media for neurons. It is preferred that the supplements in this case be prepared into a solution state before being added.

The culture medium of the present invention induces proliferation and differentiation of Arctic ground squirrel hippocampal neuronal stem cells, which are one type of the undifferentiated stem cells.

The material of the culture plate, dish, etc. is not particularly limited, and may be glass, plastic, etc. The plate or dish is optionally coated with a single layer or a plurality of layers of polylysine, polyornithine, polyallylamine, protamine, laminin, collagen, gelatin, fibronectin, vitronectin, tenascin, or a mixture of them.

### WORKING EXAMPLES

### Culture of Adult Arctic Ground Squirrel Stem Cells

### Present Status of Stem Cells:

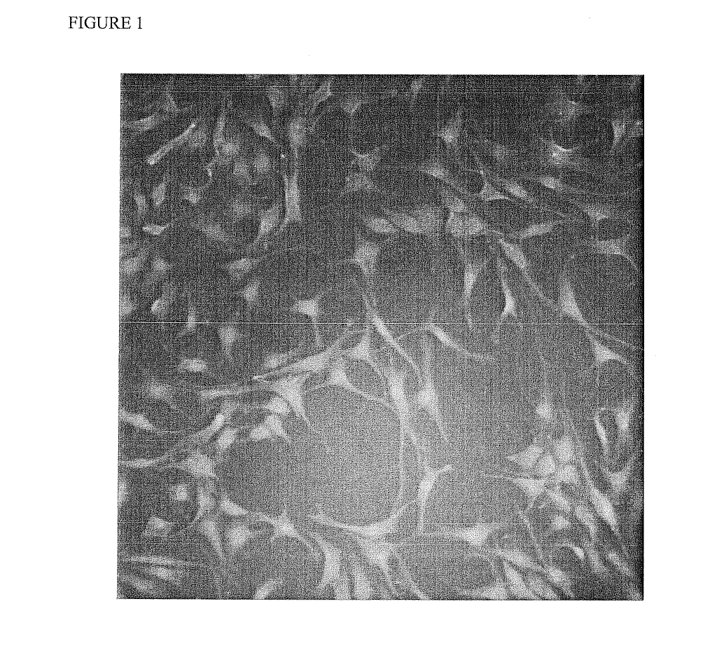

Adult hippocampal Arctic ground squirrels (AGS) neural stem cells were obtained from two (2) euthermic animals. Stem cells from one animal were cultured to Passage 13. Slight division slowing was observed beginning by Passage 12. The doubling rate is less than 24 hrs even at Passage 13. NNI has frozen stocks of cells at each passage. Enough cells were generated and frozen to make at least lots of 200 vials at 400,000 cells/vial. The seeding of 200-500,000 in a T75 flask will allow for millions of cells to be produced in four days. We have stained the neural stem cells for Nestin (P3) and have stained the early differentiated neuronal precursor with an antibody (TUJ1, Covance; P3). FIG. 1 depicts nestin-positive AGS hippocampal neural stem cells. Stem cells were fixed and then stained with the nuclear dye Hoechst (blue) and anti-Nestin (green). The majority of cells are Nestin-positive providing proof that they are mainly neural stem cells.

### Neuronal Differentiation of Adult Hippocampal AGS Neural Stem Cells:

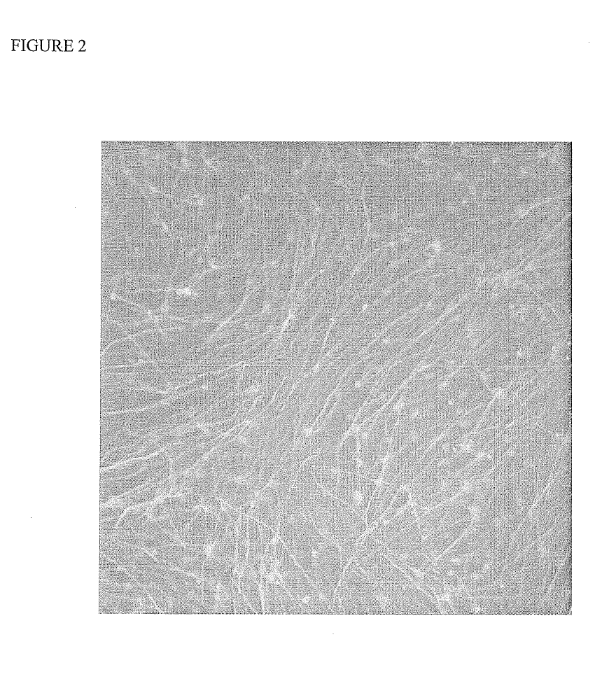

AGS Stem cells have been differentiated at a number of passages including Passage 13. Neurons have been stained with a neuron specific Class III β-tubulin neuron antibody. FIG. 2 shows that the neuronal stern cells differentiate to neurons under differentiating conditions and are neuron specific Class III β-tubulin positive.

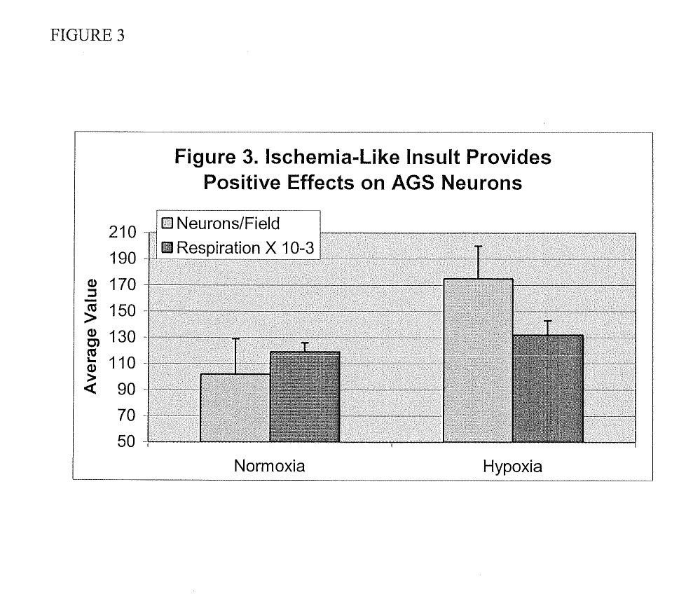

Adult Arctic ground squirrel neuron cultures were treated under hypoxic conditions (less than 1% oxygen) and under low glucose conditions (oxygen glucose deprivation, OGD) to mimic ischemia. The neurons were tolerant of this OGD treatment as determined using Alamar Blue dye of cell respiration and a Cellomics Arrayscan determination of cell and neuron numbers in each treatment well. The OGD-treated cells were similar or more active than the normal oxygen condition (normoxia) treated cells, as shown in FIG. 3. These same OGD conditions were toxic to neurons differentiated from human neuronal progenitor cells in culture.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are incorporated by reference in their entirety.

1. A composition comprising neural stem cells obtained from adult Arctic Ground Squirrel brain tissue, wherein the brain tissue is hippocampus or cortex.

2. The composition of claim 1, comprising neural stem cells obtained from euthermic or hibernating Arctic Ground Squirrel brain tissue.

3. The composition of claim 1, wherein said composition comprises neural stem cells that are differentiated to neuronal progenitor cells and neurons.

4. The composition of claim 2, wherein the neuronal progenitor is in the form of a transfected cell to be transplanted or used for identifying therapeutics for neurological disorders and injury.

5. The composition of claim 2, wherein the neuronal progenitor is transplanted or used for identifying therapeutics for neurological disorders and injury.

6. A method of culturing the neural stem cells of claim 1, said method comprising:
obtaining Alaskan ground squirrels (AGS) neural stem cells from euthermic or hibernating animals, and
developing a medium for maintaining the stem cells, wherein the developed medium is sufficient to provide nutrients for optimum growth and to induce neuronal progenitor cells and for differentiation to neurons.

7. A method of culturing the neurons differentiated from AGS neural stem cells of claim 1, said method comprising:
developing a medium for maintaining the neurons, wherein the developed medium is sufficient to provide nutrients for optimum function and maintenance.

8. An antibody specific for Arctic ground squirrel neural stem cells.

9. A kit comprising the antibody of claim 8, at least one container for reagents, and directions for use.

10. A cell line obtained from the composition of claim 1, wherein the gene target or targets for ischemic tolerance in Arctic Ground Squirrels has been knocked out.
